(19) United States
(12) Patent Application Publication
(10) Pub. No.: US 2023/0138052 A1
(43) Pub. Date: May 4, 2023

(54) Disposable Comprising A Security Element, Security Element and Method for Protecting, by the Manufacturer, Permissible Usage

(71) Applicants: Fresenius Medical Care Deutschland GmbH, Bad Homburg (DE); Fresenius Medical Care AG & Co. KGaA, Bad Homburg (DE)

(72) Inventors: Jonas Hellhund, Frankfurt (DE); Robert Lindemann, Wiesbaden (DE); Arne Peters, Bad Homburg (DE); Gerome Fischer, Weberstedt (DE)

(21) Appl. No.: 17/432,362

(22) PCT Filed: Feb. 19, 2020
(86) PCT No.: PCT/EP2020/054291

§ 371 Date: Aug. 19, 2021

(30) Foreign Application Priority Data
Feb. 20, 2019 (EP) 19158314.5

(51) Int. Cl. G06F 21/44; G06F 21/73

(52) U.S. Cl. CPC G06F 21/44; G06F 21/73; G06K 19/10

(57) ABSTRACT

The present disclosure relates to disposables for a usage application of a consumable in a medical apparatus, comprising a security element to protect against tampering or misuse by unauthorised third parties, wherein the security element has a first identification marking which materially forms a first manufacturer's identification code; the first identification marking is connected in a non-detachable manner to the disposable and is configured for uniquely identifying the disposable, such as with regard to a product type and/or product usage property, and is partially or completely concealed with a concealing means which is detachable in a non-destructive-free manner. Furthermore, methods for marking a disposable, methods for enabling a disposable in an associated consumption device, and methods for system-control of both aforementioned methods are proposed. Furthermore, the disclosure relates to associated control systems or associated computer programs for performing an aforementioned method, and to security elements.

### CROSS-REFERENCE TO RELATED APPLICATIONS

The present application is the national stage entry of International Patent Application No. PCT/EP2020/054291, filed on Feb. 19, 2020, and claims priority to application Ser. No. 19/158,314.5, filed in the European Patent Office on Feb. 20, 2019, the disclosures of which are expressly incorporated herein in their entireties by reference thereto.

### TECHNICAL FIELD

The present disclosure relates to disposables for a usage application of a consumable, e.g., in a medical apparatus which is provided with a security element, and to security elements for applying to a disposable. Furthermore, the disclosure relates to methods for marking a disposable, furthermore methods for enabling a disposable in an associated consumption device, such as a medical apparatus, and to methods for system-control of both aforementioned methods. Furthermore, the disclosure relates to associated control systems or associated computer programs for performing an aforementioned method. The present disclosure relates to possible ways of the manufacturer protecting a disposable against being tampered with or misused by unauthoised third parties, and of safeguarding application-critical properties of the consumable in the associated consumption device against impermissible usage. For example, in the present case, it is possible for the manufacturer to safeguard at a usage location that a specific product type of a dialysis module is used and activated only in a permissible or compatible manner, e.g., only for a diagnosis specific to a patient and/or in an associated dialysis apparatus.

In the prior art, e.g., in the medical sector requiring sterility which is to be absolutely maintained in the daily routine of a clinic, so-called disposables in a wide range of forms and designs are commonplace. Furthermore, with medical advances it is known to provide possible ways of effecting at least partly automated monitoring and protection of the provision or usage of such disposables to ensure that in any event, a patient receives only a suitable treatment or specifically the treatment ordered by a doctor in each case. In terms of at least partial automation of the protection measures to avoid human error, and of increased efficiency of clinical procedures, it is also known to employ technology using apparatus-readable codes.

For instance, DE 10 2016 221 337 discloses a medical treatment system comprising a medical treatment apparatus which is arranged to select a treatment and to generate a patient-related and treatment-related request code, and comprising a therapy enabling apparatus for receiving the request code, also comprising an enabling code generation device. The medical treatment apparatus decides, at least in dependence upon the enabling code and possibly the previously generated request code, whether and, where appropriate, how the requested treatment is enabled.

Proceeding from the aforementioned prior art, an advantage of the present disclosure is to provide or improve, with regard to individual disposables of a consumable, possible ways of ensuring a usage application, which is correct or permissible specifically for a respective consumer, at the usage location. A possibility of an incorrect or impermissible usage is to be specifically recognised and ideally is also to be prevented beforehand. By reason of the recently increased diversity of patient-specific, individual treatment requirements or prescriptions and by reason of the increased complexity of clinical procedures in treatment centers and in the everyday life of, frequently ambulant, patients, the requirements for ensuring or monitoring a permissible, safe usage of disposables have recently increased.

One aspect is to provide a protection option which is complete from the point of view of the manufacturer as the market participant responsible for the product and as the guarantor, and/or which is safe from misuse, after the disposable leaves the manufacturer's direct area of influence, i.e., ex works. Codes which have hitherto been used on original disposables have the disadvantage that they can be easily read-out and copied by third parties acting without authorisation, i.e., criminals such as product pirates or counterfeiters. Then, such codes which are acquired illegally can be attached to unauthorised, unsuitable external products, attached to impermissibly reused original disposables, and/or used for prohibited enabling of treatments or for usage of equipped devices, such as a medical apparatus. Therefore, a problem to be solved is that of precluding targeted tampering and/or misuse, i.e., protecting against a bypass option with not inconsiderable dangerous consequences for the consumer. In other words, the disclosure primarily provides the advantage of guaranteeing usage of an original disposable which is in accordance with regulations and can be specific for a respective consumer, as well as ensuring an exclusively authorised use. Moreover, the further advantages of the disclosure are those of improving the production and delivery chain of disposables, as well as providing a disposable in an efficient and error-proof manner at a usage location and/or to unburden the persons involved in a usage application, e.g., in the medical sector when treating a patient.

These aspects are achieved by subject matter as described herein. Advantageous embodiments, developments or variants are the subject matter of the description and the figures.

According to a first aspect, a disposable is provided for a usage application of a consumable, e.g., for usage in a medical apparatus, comprising a security element to protect against tampering or misuse by unauthorised third parties. The security element comprises a first identification marking which materially forms a first identification code. The first identification marking is connected in a non-detachable manner to the disposable and is configured for uniquely identifying the disposable, such as in terms of a product type and/or product usage property of the disposable. The first identification marking is partially or completely concealed, e.g., visually concealed, with a detachable concealing means.

In the present case, the term “disposable” relates to an individual unit or an individual piece. In the present case, a disposable is used or consumed individually by a consumer, such as a patient, and/or by a user, such as a doctor. A disposable is an individual part of a larger entity of a consumable. “Consumable” means a product or article which is produced as such mostly in large quantities and/or can be purchased on the market. A disposable relates, on the one hand, to one-time uses, i.e., a single-use article, and optionally with additional packaging or secondary packaging, e.g., a sterile packing unit. A disposable relates, on the other hand, to recyclable or re-usable disposables for multiple-use applications, such as, e.g., module units which are returned to an original state for further use by means of reconditioning, performed mostly by the manufacturer, and/or cleaning and/or sterilisation and/or functional refurbishment after use. By way of example, module units are considered to be filter inserts, e.g., hollow fiber modules. Disposables comprise purely physical usage accessories and/or primary packaging or applicators which contain, e.g., medical, chemical and/or pharmaceutical preparations, formulations, or dosages, e.g., of all aggregate states. Primary packaging is thus all packaging means which come into direct contact with the actual consumable, e.g., films, glass, containers, lids. Primary packaging or applicators can be manufactured from the most varied product-resistant materials, e.g., from synthetic material, such as polyethylene, polypropylene, polystyrene, from metal such as high-grade steel and/or aluminium, from glass, from porcelain or from composite materials, e.g., multilayer films. In the medical sector, primary packaging or applicators are often designed having multiple compartments for increasing the shelf life of an included consumable, which is provided having multiple compartments. Disposables, also in the case of one embodiment as primary packaging, such as an applicator, are frequently individually additionally packaged, e.g., with a transparent film, such as a film bag, sachets, or flow packs. An additional packaging means is secondary packaging, irrespective of whether the disposable additionally packaged therewith comprises primary packaging or not.

In other words, the disposables can be used as such independently on one occasion, such as infusion bags or catheter kits. Additionally, the disposables, in the case of an entire technical system, can be provided in an associated consumption device, such as in a medical apparatus for modular use. An example of an entire system relates, as a disposable, to a dialysis module having product-specific dialysis application properties, e.g., with regard to substance separation, which is used in an appropriate dialysis apparatus of a dialysis center for a specific patient as a consumer. In this case, haemodiafiltration, haemodialysis, and haemofiltration are referred to as variants of dialysis applications. Hollow fiber modules can depict such variants by corresponding operation modes or by means of corresponding features of their hollow fiber membrane. Different operation modes can be varied by corresponding control of a machine in which the hollow fiber module is used. Furthermore, connections of feed lines and discharge lines on the machine can be changed. A further example of a disposable item is a membrane and/or a filter for use in a reverse osmosis unit.

Insofar as the disclosure is aimed at safeguarding an initial use of the disposable, or in the case of an accessory unit a start-up operation, only by authorised persons, it is not essential whether the disposable is completely consumed or used up. Therefore, this expressly also includes embodiments, in which, after the first-time authorised commencement of usage, the usage is ceased with a residue left over or is interrupted and optionally resumed in a suitable manner.

As mentioned above, the security element described herein comprises a first identification marking which materially forms a first identification code. Both an identification code and a respectively associated identification marking serve as identifiers for the same disposable. That is, both terms correspond to one another, wherein merely for the sake of improved clarity the respective design is to be differentiated, i.e., whether it is virtual or information technology-related, or material, tangible, or physical. For example, an identification code is a serial number which is consecutive in production for the individual disposables of the consumable and for which an individually allocated code is specified, which can be converted into an encrypted and/or coded form. This serves to uniquely identify the individual disposable by its associated identification marking which forms the identification code, which is present mostly in numerical form, i.e., in the virtual space, in the material space or physically, e.g., displays said code in a visual manner or reproduces it such that it can be read (out). In the present case, the term “identification marking” (or “label”) refers to an identification or provision of a unique identification option, e.g., lettering, a sign, a tag, a sticker, an imprint, a stamp, a signature, a transponder, a chip, etc. It is a form of identification marking carried out by the manufacturer which is not visible and which cannot be followed up by an external third party in terms of information content.

Within the scope of the disclosure, the term “identification code” or “identification marking” can apply to all of the codes known in the prior art and typical standards, but also future ones. The latter can be achieved over the course of time with new code technologies, such as code generation and/or code detection methods in accordance with underlying physical principles. Therefore, in the present case, the terms “identification code” or “identification marking” are to be understood analogously and functionally. As far as clearly visible identification markings are concerned, e.g., single-row or multiple-row so-called bar codes are established. For example, it can be a so-called “codablock” (an internationally applied two-dimensional bar code). In one embodiment, a marking in accordance with the HIBC standard, which is known as a special form of bar code (HIBC: official abbreviation for health industry bar code; different standards of automatic data communication which are intended to ensure reliable product identification and retracing worldwide within the health industry). Another identification marking which is routine in practice at the time of the invention is introduced as the “RFID label” (radio-frequency identification, i.e., “identification with the aid of electromagnetic waves”) and designates a technology for transmitter-receiver systems for the automatic and contactless identification and locating of objects on the basis of radio waves.

As mentioned above, the first identification marking described herein is connected in a non-detachable manner to the disposable and is configured for uniquely identifying the disposable, such as in terms of a product type and/or product use property of the disposable. In other words, the first identification of the disposable according to the first identification marking or the first identification code comprises a variety of characteristics and/or master product data relevant thereto, e.g., technical product data and other information such as production-related and/or application-related specifications, a model series, an individual production time or production shift, a production site, a production facility and/or production installation(s), one or more raw materials or semi-finished goods, a batch number, check digit, check measurement protocol etc. Application-related product usage properties can be, e.g., data relating to dialysing fluids (type and composition of the dialysing fluid and/or substitution fluid), dialysers, single-use articles used, such as tube kits, dialysis filters, etc. In general, the production-related and/or application-related data include at least so many characteristic features that a use or treatment is to be allocated uniquely to a consumer, such as a patient. In the context applied in this case, the first identification marking which functions as a first identification corresponds quasi to an ‘identity card number’ or a fingerprint for a respective disposable. The first identification is intended to provide an ability to identify a disposable from the beginning in the form of an identity allocation to it until the end in the form of its use. Furthermore, in respect of a use of the information going beyond this in terms of time, the first identification can be used, e.g., for storing further processes.

As mentioned above, the first identification marking as described herein is partially or completely concealed, e.g., visually concealed, with a detachable concealing means. The at least partial concealment of the first identification marking provided by the manufacturer ensures that the part of said identification marking which is concealed by the concealing means cannot be recognised or detected by third parties, e.g., unauthorised persons. If a part of the first identification marking remains unconcealed, the openly accessible part can typically be used by anyone along the supply chain, from the manufacturer, e.g., in a laboratory journal and/or logbook, to a user and/or consumer and possibly also decentralised further participants and/or in a patient file. The term “concealing means” is to be understood to refer to all means suitable for concealing, rendering invisible, rendering unrecognisable, and/or rendering unusable the identification marking (in the covered region), e.g., a sticker, an applied cover layer, a film, and/or an imprint, e.g., a visual cover, and/or in the case of a transponder identification, an interference signal transponder or means for detuning a resonance circuit.

It may be that the concealing means is designed such that it can be detached in a non-destructive-free manner from the first identification marking. The phrase “in a non-destructive-free manner” relates merely to the concealing means which is to be detached from the first identification marking or separated therefrom, but specifically not to the identification marking, the integrity of which must remain, at least substantially, unaffected in terms of further usability, as a result of reversing the concealment. This means that the concealing means, e.g., a materially configured concealing means, loses its further functional usability in the detachment procedure or as a result thereof. One non-destructive-free embodiment ensures that the concealing means can be detached irreversibly or only on one occasion. In other words, a detachment ensures that it is not possible to re-attach the concealing means. For example, this can be designed in the form of a sticker which can be pulled off and has at least one predetermined breaking point, such as a perforation, and/or in the form of a scratch-off panel. Alternatively or cumulatively, it may be sufficient that an adhesive or sticking strength required for concealing and/or the inner material structure of the concealing means is greatly reduced after being detached on one occasion such that the concealing means could not be repositioned, and/or at the very least, this could be easily recognised or confirmed and ideally would be very noticeable.

A part of the first identification marking which is partially unconcealed and, e.g., can be read (out) in a visually simple manner, can be used for logistics purposes, such as for the tracking or traceability of the method steps and transport routes along the supply chain. After concealing, a partially unconcealed part of the first identification marking can then be used in the usual manner for the purpose of providing clear documentation, e.g., as part of the so-called good manufacturing practice (GMP) which is required in the medical and pharmaceutical sectors. The person skilled in the art understands this to be directives for the quality control and auditing of the production sequences and environment in the production primarily but not exclusively of medicinal products and active ingredients (see, e.g., currently valid, legal EC Directive 2003/94/EC laying down the principles and guidelines of good manufacturing practice in respect of medicinal products for human use; also the ISO 9001 standard for auditing quality management systems; in the USA the requirements of the FDA).

In one embodiment, both the part of the first identification marking which is concealed by the concealing means and the unconcealed part thereof each separately forms a meaningful graphical identification code.

In one embodiment of a first identification marking in the form of the above-mentioned “codablock” and/or in an embodiment including multiple rows, incomplete and only partial concealment can be effected in a manner such that only some of the rows provided are concealed by the concealing means, whereas the rows which continue to be visible can be used as the unconcealed part by anyone after marking has taken place. This quasi integrally designed embodiment is particularly efficient, in that inter alia continued compatibility with already existing logistical devices is also achieved in a particularly simple manner.

In the case of a further embodiment, the concealed part and unconcealed part of the first identification marking is illustrated in accordance with the industrial standard “Code 49”. “Code 49” refers to a designation introduced for a multi-row bar code for increased safety requirements as in aviation, comprising a so-called parity row, by means of which the parity can be verified for each character. Furthermore, with respect to “code 49” reference is made to the disclosure of the precise “code specification” available from the “Association for Automated Identification and Mobility”. In this embodiment, at least the last row which contains the check character for the entire block has, in this case, a row which encodes a different content adhered over it and the check digit for the block, over which the row is adhered, is adapted accordingly to obtain the code integrity of the marking.

In the case of a still further embodiment, the concealed part and unconcealed part of the first identification marking are encoded in a so-called “composite code”, which in the present case is intended to signify that in the two parts in each case, a link or cross-reference to the other part can also be encoded. For example, the composite code can also be configured as a double code symbol, as in the form of a bar code composed from a linear. i.e., 1-dimensional bar code and an additional 2-dimensional code.

In contrast to a merely partial concealment, in another embodiment, specifically a complete concealment of the first identification marking may be desired in order to produce a hidden identification marking. Such a hidden design provides particular protection against forgery activities by unauthorised third parties. For example, the first identification marking can be provided at a position which is inaccessible in the original state of the disposable.

In one embodiment of the disposable, the disposable also comprises a second identification marking which materially forms a second identification code. The second identification marking is not concealed, e.g., it is clearly visible and is arranged for an at least temporary logistical use, such as traceability. This embodiment includes as possible variants that the second identification marking is not attached exclusively, or is not attached at all, on the individual, optionally individually originally additionally packaged, e.g., film-packaged, disposable itself, but instead is attached on a plurality of disposables, such as a twin pack, multi-pack or large pack and/or a transport container, e.g., cardboard boxes etc.

The second identification marking is part of the concealing means, that is to say that a separate concealing means for concealing the first identification marking is not required, insofar as the first identification marking is concealed directly by the second identification marking. A part of the concealing means can also mean that materials are identical, that is to say that the second identification marking and the concealing means coincide as a unit. For example, this can be effected in the form of two layers of a first identification marking and a second identification marking applied one on top of the other. In this embodiment, it may also be that the concealed part of the first identification marking, which is concealed with the second identification marking, and the completely unconcealed second identification marking constitute, again in combination, a third identification marking having a meaningful, associated, third identification code resulting from the superposition. As a result, particular error correction mechanisms of the marking or encoding can be implemented. Alternatively, parts of the concealed part of the first identification marking can be quasi ‘deleted’ by the colour of the underlying substrate. This allows increased degrees of freedom when encoding the unconcealed part of the first identification marking.

According to a second aspect, a method is proposed for marking a disposable for a usage application of a consumable, e.g., for use in a medical apparatus, to protect against tampering or misuse by unauthorised third parties. To avoid repetition, reference is made to the definition of terms given above. The features, combinations of features and the advantages resulting therefrom are provided, corresponding to those which have been performed previously in conjunction with the aspect first stated. In a first step, a first identification code is generated using information technology or is produced using computer technology and is stored by a manufacturer of the consumable. Therefore, the identification code is stored on a storage medium, e.g., in a database, within the area of influence of the manufacturer who manages and monitors the access rights to the data and can keep the data internally and confidentially. In a subsequent step, the disposable is marked with a first identification marking which materially forms the first identification code. This step is associated with a further step in order to produce a unique way of identifying the disposable with its characteristics and features which are relevant to a subsequent consumer and/or user. And furthermore, a product type and/or product usage property of the disposable is allocated, in parallel at an information-technology level, to the first identification code, whereby a first data relation is formed. This transaction or information relating to the step, e.g., the first data relation, is stored by the manufacturer. In other words, data, e.g., in the form of data packets (keyword: “digital factory”), which are allocated to the production process and/or the logistics process and which represent information about which disposable can be characterised by which product type and/or product usage property as being relevant in terms of application, are linked together in the step of the first marking, are allocated by information technology in a function, or are stored behind the first identification marking on the identification code level. This unique linking of information during the first marking procedure ensures that at each, even much later, point in time and also at another location, the characteristic information stored under the first identification code and relating to the respective disposable, e.g., a product type and/or product usage property, specifically relating to a use of this first data relation can then be used and made available by the first identification code. Insofar as the first data relation is in the possession of the manufacturer, such a reversing step or the inference from the first identification code to the information linked in the first data relation, can only take place with the involvement of the manufacturer, i.e., with his authorisation.

In a further step, the first identification marking is concealed with a concealing means. The first identification marking is partially or completely concealed, e.g., it is visually concealed. Furthermore, the concealing means is applied so as to be detachable from the first identification marking. This combination of features ensures that until the detachable concealing means is removed, the first identification marking remains concealed for one use, therefore the first data relation cannot be used in the aforementioned manner by anyone up to this point in time via the identification code.

For example, when concealing the first identification marking, only individual graphical elements of the code representation are covered by an ink, and also, for example, on a detachable carrier, so that the unconcealed graphical elements form a new, meaningful, identification code-representing identification marking.

For example, complete concealment can be advantageous if the concealing means and, underneath it, the first identification marking remains entirely hidden in a suitable discrete embodiment of the disposable in order not to give unauthorised persons, such as forgers, any idea of possibly attempting to use the disposable after tampering with it in some way.

Alternatively, specifically a partial concealment may be used in order to illustrate a particularly effective, quasi integral embodiment of security element for a disposable. This is because in the case of a partial concealment, e.g., by a partial-region of a multi-row bar code, such as a codablock standard, a particularly advantageous combination of a first concealed partial region of the first identification marking and a second unconcealed partial region of the first identification marking is produced. In this respect, the unconcealed partial region of the first identification marking can be used to form or graphically represent a separately contained identification code, e.g., for the purposes typical for identification codes, e.g., handling and tracking. The concealed partial region of the first identification marking forms, in its own right and/or in combination with the unconcealed partial region, the first identification code used for identification and protection.

Optionally, the concealing means is configured so as to be detachable in a non-destruction-free manner. This ensures that—as with a sealing wax on a letter—the first identification marking can only be rendered visible once, and then anyone can clearly see that it is in a non-original state.

Depending on this, based on various technical considerations, e.g., for practical reasons relating to the layout of the production factory and the production installations specifically used, but also relating to the material flow and logistics, including storage, different variants of the method steps of first marking and concealing in the corresponding method stage(s) may be used: concealing can take place following on from the first marking, e.g., in a synchronised, linear continuously operated installation having a conveyor belt, by applying a first identification marking at an upstream installation stage, and then applying a concealing means at a subsequent further installation stage. In another embodiment, the method may include applying an embodiment of a prefabricated security element to the disposable in the first marking step. The prefabricated security element comprises a combination of an identification marking which is already previously concealed with a concealing means, e.g., a composite layer. In such continuous production process control described above, the prefabricated security element is produced on the production line initially at an upstream stage and/or is fed to the production line from a store.

In the present case, the term “manufacturer” (or “by the manufacturer”) refers to a company which wishes to guarantee or has to legally guarantee the specific properties of its consumable when used by the consumer. For example, in the case of processes subject to auditing, e.g., in the field of medical products, the manufacturer is a company which is qualified to produce or market the consumable, is an authorised company, or is a company licensed by authorities which are to be involved. In its local sense, the term includes the national and international locations for production, storage, and/or logistics. Likewise, within the scope of the present invention this includes not only manufacturing areas, but also primary and secondary packaging lines. Furthermore, in the present case, the term relates not only to the original manufacturer in the narrowest sense, but also to a contract partner, a sub-contractor, a certified contract manufacturer, and/or logistics service provider acting on behalf of and/or for the origin company. For instance, it may be the case that a production and/or packaging business process stage in advance of the delivery of the shipment of goods (in the form of the present disposables, ex works and/or warehouse (the so-called “dispatch”)) to the consumers or users is allocated externally in a qualified manner (“outsourced”). With regard to an information technology service to the manufacturer, the phrase “by the manufacturer” likewise includes those, primarily contractually bound, service companies. For example, the scope of the invention includes the sub-case that an identification code is produced in advance by a separate information technology service provider and is passed on to the manufacturer for its use.

The term “information technology” is to be understood to be a technical term and relates to the computer-based or computer-implemented execution of steps which can also be implemented by means of software and/or hardware. Generating and allocating using information technology can be performed, e.g., by corresponding algorithms and/or applications. The respective algorithms and/or applications can be executed locally and/or remotely (e.g., on a computer entity which exchanges data). Moreover, the corresponding algorithms and/or applications can be provided in a virtualised form. Therefore, “information technology” relates to an automatic and ultimately processor-based implementation using integrated circuit modules.

The term “data relation” is based upon a mathematical mapping rule (or function, allocation). In the present case, it can be a bijective or biunique data relation. It is essential that, e.g., proceeding from the first identification code, an identification, e.g., a reverse conclusion to a product type or product usage property which is application-relevant for the enabling is permitted or arranged.

In one embodiment, the method for marking comprises further steps: generating, using information technology, a second identification code and storing by the manufacturer, marking the disposable with a second identification marking and materially forming the second identification code, wherein the second identification marking is applied in a unconcealed, clearly visible manner; and allocating, using information technology, the second identification code to the first identification code to form a second data relation and storing by the manufacturer.

In an embodiment, the first identification code and the second identification code can be generated as parts of a further identification code which is superordinate in terms of information technology. Alternatively, it may also be the case that the first identification code and the second identification code are entirely independent of one another. The first identification code and the second identification code can be generated in a different manner. Irrespective of this, the first identification marking and the second identification marking can be configured according to a different type or according to another technical principle and/or can be applied to the disposable in a different manner. For example, a first identification marking can be printed onto the disposable as a bar code and the second identification marking can be adhered in the form of an RFID label.

In one embodiment of the method for marking, the second identification marking for marking the disposable is part of the concealing means. A part of the concealing means can also mean that materials are identical, that is to say that the second identification marking and the concealing means coincide as a unit. Therefore, the first identification marking is partially or completely concealed in a very efficient manner by omitting an additional concealing means.

In one embodiment of the method for marking, the marking with the first identification marking is effected according to the following variants which are all to be understood to be alternative and/or cumulative: initially, as already stated for the concealing means, it can likewise be advantageous for the first identification marking to configure its connection to the disposable in a form which is detachable in a non-destructive-free manner, in order to make it possible to recognise an original state. The first marking procedure can be performed by means of punching, lasing, printing, embossing, plating, coating, inlay methods, fixed adhesion, 3D printing and/or sealing which constitute industrially reliable and effective marking methods which are widely adaptable in terms of production technology. In terms of a meaningful layout of the production of the disposable, the first marking procedure can be integrated into a quality control stage and/or into a packaging stage of the production of the disposable. For example, insofar as multiple consumables can already be packaged in-line, quasi as a final production step, one variant may include performing the first marking procedure on respective additional packaging of the packaging unit. This variant may be used if the additional packaging can be added to the disposable, e.g., in the case of additional packaging ensuring a necessary level of sterility. In a further alternative or cumulative variant, the first identification marking is clearly visible under transparent respective additional packaging of the packaging unit, such as a transparent film. The connection of the first marking can be configured in a non-detachable manner as a lacuna at least in an originally packaged and/or originally closed state of the disposable. Such an integral connection provides the advantage that, by means of the disposable, it would be difficult for any attempt at stealing identity for unauthorised purposes to remain unnoticed. Furthermore, the first identification marking can be provided in a region of the disposable, such as inside a closure cap, which is concealed at least in the originally packaged and/or originally closed state and/or is not externally accessible. This serves to further hinder possible misuse or tampering, insofar as the first identification marking is hidden up to the point in time a closure is initially broached or opened.

In one embodiment of the method for marking, at least one of the two steps of concealing with the concealing means and of marking with the second identification marking is performed in a variant to be understood as cumulative and/or alternative: the concealing means applied to the first identification marking and/or the second identification marking applied to the disposable is detachable in a destructive-free manner for the first identification marking. Production methods for forming a detachable connection constitute reversible surface methods and/or multiple layer methods, by means of applying an adhesive cover layer, a cover layer which can be scratched off, an intermediate separation layer, a detachable adhesion layer, and/or by means of printing with reversible printing ink. In this manner, robust production processes can be operated and the production stage can be integrated onto machines which may be present. At least one of the aforementioned two method steps can be performed in a quality control stage of the production of the disposable and/or in a packaging stage of the production of the disposable. This provides for the most integral possible configuration of the factory layout and associated production sequences. Additionally, at least one of the steps is performed in-line directly following on from and/or in an integral method stage with the step of marking with the first identification marking. The concealing means applied to the first identification marking and/or the second identification marking applied to the disposable can be applied on respective additional packaging of the packaging unit and/or in a clearly visible manner under transparent respective additional packaging of the packaging unit. This increases the degrees of freedom of product design and production or packaging sequences. This can be of particular benefit, e.g., in those cases where national requirements provide particular and particularly complex legal directives relating to the marking of consumables which have to be marked, with implications for the packaging sequences, e.g., in the form of specific package information sheets or primary packaging in the respective official language of a respective country. For example, the second identification marking can be provided in the form of a label which can be used for logistics purposes. For example, it can be an RFID label and/or a label designed for storing a transaction chain. In the case of concealing means which does not coincide with the second identification marking, it may be that the second identification marking is not provided on each individual disposable but instead only on a twin pack, mufti-pack, or large pack including a plurality of disposables. In this respect, the second identification marking is used mainly for dispatch purposes and it represents a variant which is particularly conserving of resources, is environmentally friendly, and is particularly flexible in terms of the point of application of the second identification marking.

According to a third aspect, a method is proposed for enabling a usage application of a disposable according to the first aspect in an associated consumption device, e.g., in a medical apparatus, including protection against tampering or misuse by unauthorised third parties, comprising the steps of removing the concealing means from the disposable at a usage location as the location of a consumer and/or a user; and authorising the usage and/or a respectively associated usage purpose of the disposable. The removal of the concealing means exposes the first identification marking such that it is recognisable and clearly visible. For authorisation, at least the first identification code is recognised. By means of the first identification code and the associated first data relation, it is possible for the product type and/or product usage property of the disposable, which are stored and saved in a manner previously allocated by the manufacturer, to be used as information in an enabling control element.

Furthermore, in one embodiment of the method for enabling, the second identification code is recognised in the step of authorising. Then, by means of the second identification code and the associated second data relation (relating to the first identification code), the information available for the first identification code, including the product type and/or product usage property or the first data relation, are also linked and can be obtained in an authorised manner via the manufacturer. By supplementing the information provided for the first identification code with the information provided for the second identification code, further conclusions can be drawn in respect of monitoring and ensuring an authorised and/or appropriate usage application. For example, the information can additionally influence a delivery chain or destination address of a consumer or user. This provides increased transparency and thus protection of the authorisation.

In an embodiment, the identification code recognition of the second identification marking, e.g., by means of a scanner, takes place in advance of and/or at the same time as the identification code recognition of the first identification marking. Therefore, this is a particularly effective, rapid form of recognition in a procedure. It may be that the first and second identification markings are detected or recognised or scanned separately.

In one embodiment of the method for enabling, at least the first identification code is read visually by the consumer and/or the user or is recognised by means of a separate code reading apparatus and is transmitted to the consumption device and/or is automatically recognised by the consumption device.

In one embodiment of the method for enabling, a connection of the concealing means, e.g., of the second identification marking, to the disposable also serves to effect the removal of the concealing means during usage of the disposable, e.g., when opening the disposable, e.g., when removing a lid or a closure cap, and/or connecting the disposable to the consumption device. This provides particularly speedy and convenient sequences during the usage application, e.g., in the everyday activity of a clinic.

In one embodiment of the method for enabling, an action of disabling the disposable for usage is provided as a further step. For instance, a disabling action can be triggered after refusal of or failure to acquire authorisation, wherein the non-compliance with a condition to be fulfilled for authorisation purposes can cumulatively and/or alternatively affect inter alia the following initiating reasons: a permissible usage purpose previously stored for the consumer and/or the user, such as, e.g., a repeat medical prescription; on the basis of a verification of the product type and/or product usage property of the disposable; an upper time limit allocated to the disposable, e.g., on the basis of a shelf-life and/or a time window after arriving at the usage location and/or after recognition of the first identification code; and/or a compatibility of technical requirements relating to the correct usage of the disposable with the technical data of the consumption device. For example, in the event of a disabling action, information relating to an initiating reason and/or solution to a problem can also be provided by the manufacturer. In one embodiment, a method for system-control for performing an embodiment of the previously described method for enabling in combination with an embodiment of the previously described method for marking is proposed. The step of authorising is performed by comparing, using information technology, the first identification code recognised at the usage location with the first identification code, which is generated and is stored by the manufacturer. For example, the comparison can be performed cumulatively and/or alternatively to preceding embodiments: with a copy of the generated first identification code previously stored in the consumption device; with a copy of the generated first identification code previously stored with the consumer and/or the user; with a copy of the generated first identification code which is previously stored in a decentralised database of an authorised further participant, e.g., a computer-based entity of a health insurance fund. For example, the comparison and/or the transmission of the previously stored copy can be performed by means of remote communication via an interface to a database of the manufacturer for managing at least the first identification code.

In an embodiment of the previously described method for system-control, the first and the second identification markings are encoded both by layer and colour with respect to the method for marking. In production, only a part of the colour spectrum and/or the additive colour mixture is recorded by the manufacturer as a mixed code. Subsequently, at the usage location, with regard to the method for enabling, the layers which form the first and second identification markings are initially separated and then both the mixed code and the first and second identification markings are each individually scanned. If the upper layer in the form of the first identification marking is removed once, it is no longer possible to reposition it exactly at the original position. Therefore, it is advantageously possible to check the disposable to establish if any tampering has possibly taken place by comparing the scan of the mixed code and the code calculation of the first and second identification markings.

In one embodiment of the method for system-control, the step of authorising includes as a further step an information-technology use of the second data relation for taking into account information and/or a transaction chain collected as far the usage location relating to the production process and logistics.

In one embodiment of the method for system-control, the step of authorising includes as a further step an information-technology use of the first data relation for taking into account the product type and/or product usage property of the disposable.

In one embodiment of the method for system-control, an information-technology step is processed and/or stored in at least one transaction chain, e.g., in a block chain with linked transactions in a database management control system configured for guaranteeing documentation security between a plurality of distributed participants. For example, this relates to one or more steps: code-generating; storing, by the manufacturer, data from production and/or logistics; allocating to form the first data relation, allocating to form the second data relation; authorising; comparing; verifying; using the first data relation, using the second data relation; disabling action; and/or information. If a database management control system is provided, it can be configured to perform the method, e.g., for the purpose of documenting a file of a participant, such as a patient file, and/or for the purpose of scheduling dispatches to usage locations, such as facilities for medical applications and/or for the purpose of monitoring.

The term “transaction chain” or a so-called “block chain” refers in general to a continuously extendible list of blocks in the form of data records to be stored. The content of the blocks represents, e.g., initial, historical, time-related and/or local master data and use data of the disposable item. The blocks can be encrypted by means of cryptographic methods. Moreover, the blocks can be chained to one another in order to form a chain of blocks. For example, a block can contain a cryptographically secure “hash” value of the preceding block, e.g., a time stamp, a signature, and/or transaction data. By storing the blocks constructively, they cannot subsequently be modified unnoticed, which ensures the integrity of the entire system or the block chain. As a result, local data manipulation of the blocks can be counteracted by means of decentralised control. In other words, documenting security is achieved over the course of time or along the data history, e.g., as far as data versions of the blocks are concerned.

For example, the term “transaction chain” also includes a distributed database management system including a plurality of participants, e.g., a documentation-secure data documentation system managed in a decentralised manner, e.g., in the field of medical applications and therapies, such as dialysis.

The term “participant” includes computer-based entities which can be allocated to different entities, namely: the manufacturer of the consumable including its raw material and component suppliers; the consumer as an end consumer, e.g., a patient; the user, e.g., a clinical or therapeutic user, such as a doctor, a nurse, a care robot; a health coordination center; and/or a logistics service provider, such as a transport company etc.

The term a “(distributed) database management control system” relates to what is generally called a “distributed ledger” which in technical terminology is frequently abbreviated to DLT, Technology, i.e., a decentralised storage system. For example, the distributed ledger is distributed in the sense that a plurality of copies of blocks of the distributed ledger are distributed (sometimes even spatially) in different storage units. The distributed ledger comprises a plurality of data records which can also be individually addressed. For example, the plurality of data records are organised as data blocks. For example, the blocks are created by different entities, e.g., different computer-based nodes of a network, and are stored on the different entities. In other words, the data records typically are not constructed and maintained by a central entity. In typical cases, all or at least a very large number of the nodes of the network keep a copy of the distributed ledger. In general, the storage and updating of the distributed ledger and the retrieval of data from the ledger is typically based upon a consensus mechanism. The consensus mechanism ensures that the different copies of the distributed ledger correspond and therefore overall tampering possibilities and/or error sources are prevented in a structural-technical manner and document authenticity and also document security of the time-variable versions of the data records are technically supported or guaranteed.

According to a fourth aspect, a control system is proposed which is configured to perform an above-described method for system-control. The control system comprises: a control apparatus and a marking device, configured to simultaneously and/or separately apply at least the first identification marking and/or the concealing means at the location of the manufacturer; and/or the consumption device, for usage of the consumable at the location of the consumer and/or the user.

Furthermore, in an embodiment the control system can comprise a database management control system which is arranged between a plurality of distributed participants and which is configured, e.g., as a block chain with linked transactions to guarantee documentation security.

In one embodiment, a computer program is provided which, when loaded into a digital memory of a computer, implements an embodiment of the above-described method for marking and/or enabling according to the second and third aspects, and/or executes a control system according to the fourth aspect.

According to a fifth aspect, a security element is proposed which is configured to be applied to a disposable for a usage application of a consumable, e.g., for use in a medical apparatus, and is arranged for protecting the disposable against being tampered with or misused by unauthorised third parties. The security element as described herein comprises: a combination of a first identification marking, which materially forms a first identification code, and a second identification marking which materially forms a second identification code. The first identification marking is arranged to form a non-detachable connection to the disposable and to provide a unique identification of the disposable, such as in terms of a product type and/or product usage property of the disposable. Furthermore, the first identification marking is partially or completely concealed, e.g., visually concealed, with a concealing means which, e.g., is detachable in a non-destructive-free manner. Furthermore, the second identification marking is detachable and arranged for temporary use, for instance for the purpose of logistical traceability. In an embodiment, the second identification marking is identical to the concealing means. Alternatively or cumulatively, the concealing means is detachable in a non-destructive-free manner.

### BRIEF DESCRIPTION OF THE FIGURES

In the following detailed description of the figures, exemplified embodiments, which are to be understood to be non-limiting, together with the features and further advantages thereof will be discussed with the aid of the drawing. In the drawing:

FIG. 1 shows a side view of a disposable marked with a first identification marking;

FIG. 2 shows a side view of a disposable;

FIG. 3 shows a side view of a disposable according to an exemplified embodiment;

FIG. 4 shows a flow diagram illustrating steps of an exemplified embodiment of a method for marking;

FIG. 5 shows a flow diagram illustrating steps of an exemplified embodiment of a method for enabling;

FIG. 6 shows a schematic view of a control system according to an exemplified embodiment comprising a disposable according to the exemplified embodiment;

FIG. 7 shows a side view of a consumable in secondary packaging comprising a plurality of disposables according to a further exemplified embodiment.

### DETAILED DESCRIPTION

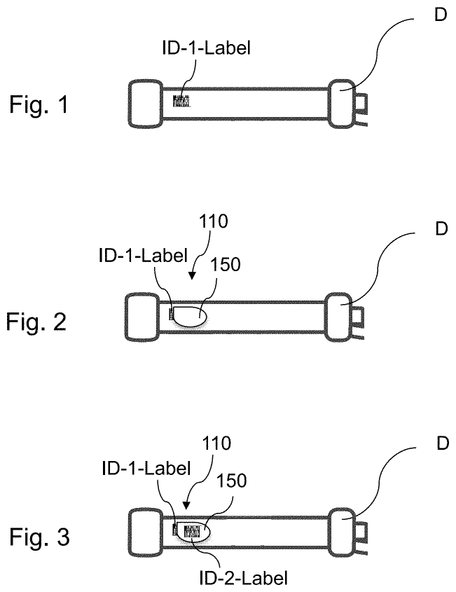

FIGS. 1 and 2 each show a side view of a disposable item for use in a medical-technical apparatus, e.g., in the form of a dialysis module D (dialyser, tube kit, etc.) which is to be used for a patient on a dialysis apparatus. The dialysis module D has a multi-row bar code imprint ID-1-Label printed on it with permanent printing ink as a first identification marking, and a side view of a dialysis module D as described herein having a security element 110 which is adhered such that it can be pulled off. The graphical illustration of the multi-row bar code imprint ID-1-Label corresponds on an information-technology side to a manufacturer's control system 400 (not shown), in which the associated numerical data code ID-1-Code is generated and also stored under a consecutive serial number as a master data information type. As can be seen in FIG. 2, the multi-row bar code ID-1-Label, with the exception of its first row, is visually concealed with a pull-off, neutrally configured sticker 150 having a square pull-off tab, thus forming the security element 110. The security element protects the masked part of the bar code ID-1-Label from being read-out or scanned. The first unconcealed row can be read-out and used in a conventional manner just like a single-row bar code, e.g., at quality-control and dispatch. As soon as the sticker 150 is removed, the state of the dialysis module then corresponds to the state prior to the concealing, as shown in FIG. 1. Then, the complete bar code ID-1-Label, i.e. all of its rows, can be read-out in a bar code scanner and can be used for identifying the individual dialysis module D.

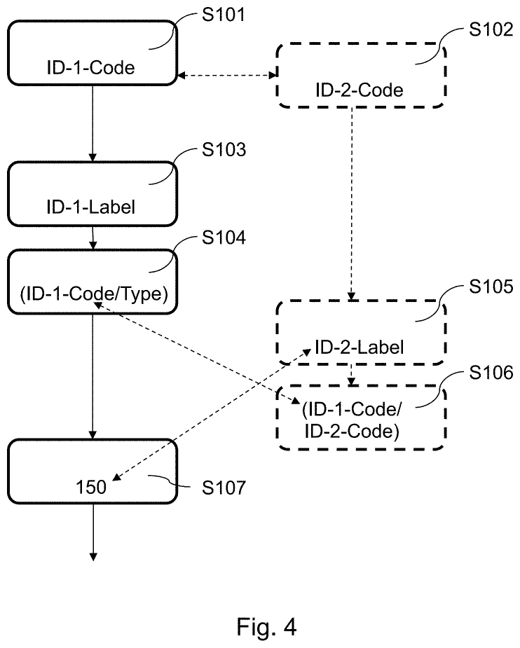

Both of FIGS. 1 and 2 likewise illustrate two production stages of a method for marking, as described below schematically with reference to FIG. 4, wherein FIG. 1 shows a semi-finished intermediate stage and FIG. 2 shows a dialysis module D comprising a security element 110.

FIG. 3 shows a side view of a dialysis module D according to an exemplified embodiment. The sticker 150 illustrated in FIG. 2 contains a second multi-row bar code imprint ID-2-Label as a second identification marking. The second multi-row bar code imprint ID-2-Label is clearly visible externally on the dialysis module D and therefore can be scanned-in and used by anyone.

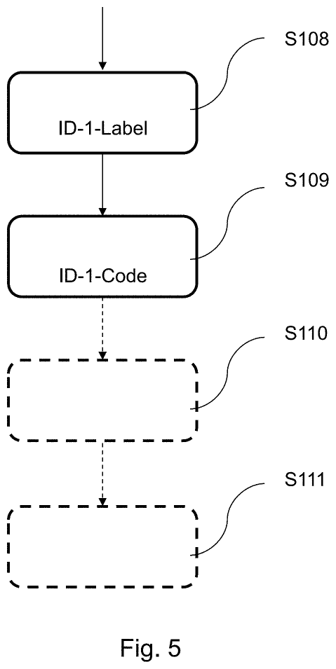

FIG. 4 and FIG. 5 each show a flow diagram which illustrates steps S101-S107 of an exemplified embodiment of a method for marking a disposable (not illustrated) for a usage application of a consumable (FIG. 4) and steps S108-S111 of a method for enabling the same (FIG. 5). Both methods serve to safeguard an identity given to an individual disposable in the form of a first identification marking ID-1-Label against tampering or misuse by unauthorised third parties. By means of the method illustrated in FIG. 4, the disposable is initially protected by a manufacturer's security element at a manufacturer's site. And after the disposable has been transported to a usage location, the method, illustrated in FIG. 5, for enabling said disposable for correct and appropriate usage for an authorised person, is performed. With regard to the schematic form of illustration, it is noted that the essential steps are represented in respective small boxes bordered by continuous lines (on the left in FIG. 4: S101, S103, S104 and S107: at the top in FIG. 5: S108 and S109). In contrast, the merely optional steps are illustrated by boxes in broken lines, insofar as they apply to some embodiments (on the right in FIG. 4: S102, S105 and S106; at the bottom in FIG. 5: S110 and S111).

The method for marking a disposable, as schematised by the flow diagram in FIG. 4, comprises the steps S101, S103. S104 and S107 which are essential: in a first step S101, a first identification code ID-1-Code is generated using information technology or is produced using computer technology and is stored by a manufacturer of the consumable. In a subsequent step S103, the disposable is marked with a first identification marking ID-1-Label which materially forms the first identification code ID-1-Code. In parallel with step S103, a product type and/or product usage property of the disposable is allocated S104, at an information-technology level, to the first identification code ID-1-Code, whereby a first data relation ID-1-Code/Type is formed. The step S104 or the first data relation ID-1-Code/Type is stored by the manufacturer. In a further step S107, the first identification marking ID-1-Label is concealed with a concealing means 150. Concealing S107 is effected partially or completely, e.g., visually, and by means of a concealing means 150 which is detachable from the first identification marking ID-1-Label.

Furthermore, the method for marking can comprise optional steps: generating S102, using information-technology, a second identification code ID-2-Code and storing by the manufacturer; marking S105 the disposable with an unconcealed second identification marking ID-2-Label; and allocating S106, using information technology, the second identification code ID-2-Code to the first identification code ID-1-Code to form a second data relation ID-1-Code/ID-2-Code and storing by the manufacturer. The double arrow, indicated in broken lines, between step S104 and step S106 illustrates that by means of the first identification code ID-1-Code, it is possible to link the first data relation ID-1-Code/Type and the second data relation ID-1-Code/ID-2-Code, whereby the second identification code ID-2-Code and the product type Type and/or product usage property are indirectly related to one another in terms of information technology. The double arrow, indicated in broken lines, between step S107 and step S105 again illustrates that, in an embodiment, the concealing means 150 and the second identification marking ID-2-Label are identical or coincide.

The method for enabling a disposable, as schematised by the flow diagram in FIG. 5, comprises the steps S108 and S109 which are essential: removing S108 the concealing means from the disposable at a usage location and authorising S109 the usage and/or a respectively associated usage purpose of the disposable. In the removing step S108, the first identification marking ID-1-Label is exposed in an (again) recognisable, e.g., clearly visible manner by removing the concealing means. The removal can be configured, e.g., as part of the procedure of opening a closure cap. For authorisation S109, at least the first identification code ID-1-Code is recognised. By means of the first identification code and the associated first data relation, it is possible for the product type and/or product usage property of the disposable, which are stored and saved in a manner previously allocated by the manufacturer, to be used as information in an enabling control element. Furthermore, the method for enabling can optionally comprise the steps of triggering or initiating a blocking action S110 and/or outputting information S111 (e.g., via a use intended without authorisation).

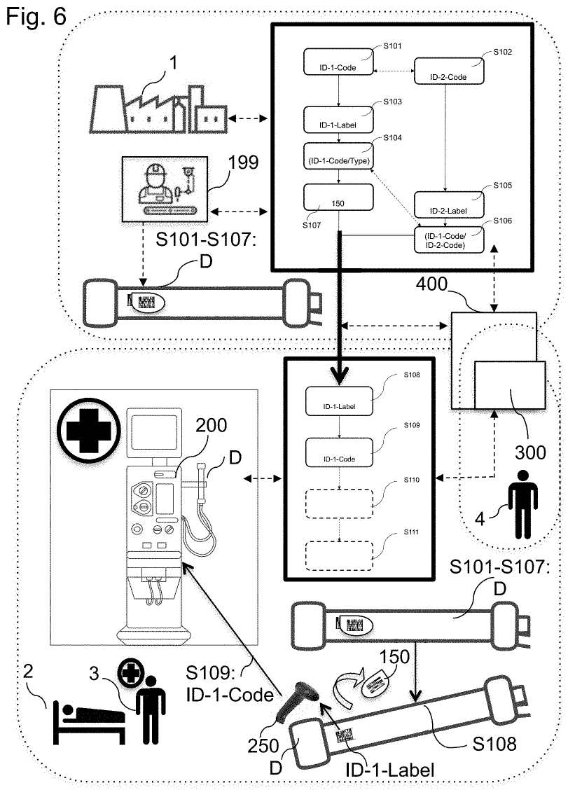

FIG. 6 shows a schematic view of a control system 400 (with a memory, not illustrated) according to an exemplified embodiment for the misuse-proof usage of an embodiment of the dialysis module D of FIG. 3 in an associated dialysis apparatus 200 as an associated consumption device, i.e., for a prescribed dialysis treatment of a patient 2 under the supervision of a doctor 3. For this purpose, the control system implements an embodiment of the method for marking (cf. FIG. 4) dialysis modules D in combination with the subsequent implementation of an embodiment of the method for enabling (cf. FIG. 5) the dialysis modules D for respectively authorised usage. The method for marking is performed in an area of influence, located at the top in FIG. 6, of a manufacturer 1 of dialysis modules D using a marking device 199. The method for enabling is performed in an area of influence, located at the bottom in FIG. 6, of the patient 2 or the doctor 3.

Furthermore, a computer-based entity of a health coordination center 4 is schematically illustrated in the center on the right in FIG. 4 as a further decentralised participant, which is configured to operate a distributed database management control system 300 (with a memory, not illustrated). The database management control system 300 is distributed, e.g., between the computer-based entities of the participants (manufacturer 1, patient 2, user 3 and coordination center 4). The database management control system 300 is part of the control system 400, with which it forms an overlapping region. The four locally distributed areas of influences of the distributed participants 1, 2, 3 and 4 are graphically indicated in the form of domains or subunits in broken lines. Broken-line arrows relating to the control system 400 with the database management control system 300 of the coordination center 4 symbolise the respective participation of the other three distributed participants 1, 2 and 3.

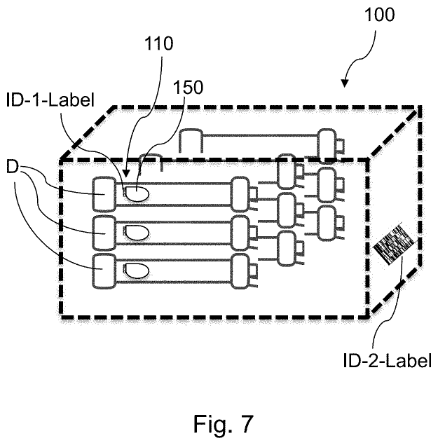

The two aforementioned methods, i.e., the method for marking using the method steps S101-S107 (cf. FIG. 4) and the method for enabling using the method steps S108-S111 (cf. FIG. 5), are illustrated in small method block diagram boxes, bordered by bold lines, in their respectively associated areas of influence, and moreover, in the area of influence of the manufacturer 1 and in the area of influence of the patient 2 and/or doctor 3. The two aforementioned small method block diagram boxes are connected from top to bottom by a transition arrow marked in bold lines. The transition arrow symbolises the combination of the two above-mentioned methods relating to a superordinate method for system-control. In the case of the method for system-control, the method for enabling follows the method for marking. In this respect, the first method step of the method for enabling S108 (at the bottom) is subsequent to the last method step of marking for enabling. In other words, an output from the method for marking in the form of the finished dialysis module D as a disposable marked with a security element enters, as an input, the method for enabling. The last method step of marking for enabling corresponds, in the embodiment illustrated in FIG. 6, to the optional method steps S105 with S106 relating to the second identification marking ID-2-Label with code ID-2-Code. After intermediate transportation of the finished dialysis module D from the manufacturer 1 to the patient 2 or doctor 3, step S108 follows which serves to render visible or ‘discover’ the first identification marking ID-1-Label. This step S108 is graphically illustrated in greater detail at the bottom on the right in FIG. 6. As can be seen, the doctor 3 uses the pull-off tab to pull the sticker 150, which is provided with the second multi-row bar code imprint ID-2-Label, off the dialysis module D. As a result, the first multi-row bar code imprint ID-1-Label (in the left region of the cylindrical middle part of the dialysis module D) is undamaged and is exposed so as to be completely visible again. Next, the doctor uses a hand-held scanner 250 to scan in the first multi-row bar code imprint ID-1-Label, whereby the associated identification code ID-1-Code is transmitted using information technology to the connected dialysis apparatus 200, which communicates with the hand-held scanner 250 (via a cable connection or wirelessly). If, with the aid of the identification code ID-1-Code, it can be positively determined (control system 400, dialysis apparatus 200, step of authorising S109) that the dialysis module D read-out by the doctor 3 can perform a dialysis treatment which is prescribed or suitable for the patient 2 and that, furthermore, the dialysis apparatus 200 set up in the relevant treatment room is configured, in terms of its apparatus design or model type, in a harmonised or suitable manner for a dialysis operation with the dialysis module D as an apparatus system, the dialysis treatment is commenced with authorisation. In the other case of a negative determination, in step S111 information relating to solutions to problems can optionally be output on the display screen of the dialysis apparatus; e.g., a notification to the doctor 3 that a further dialysis apparatus corresponding to the read-out dialysis module D is located in another treatment room or that the shelf-life of the dialysis module D has been exceeded, etc. In the case of a positive determination or after the dialysis treatment has been performed, such information or a transaction can be relayed to the control system 400 or to the database management control system 300 for resolving further optional functionalities. FIG. 7 shows a side view of a consumable 100 in a cube-shaped dispatch box (outlines indicated in broken lines) as secondary packaging with nine dialysis modules D as disposables according to a further exemplified embodiment. Each of the nine dialysis modules D corresponds to the embodiment illustrated in FIG. 2 and comprises in this respect a security element 110 having a multi-row bar code imprint ID-1-Label which is masked except for the first row by a sticker 150. Irrespective of, e.g., the forwarding agent, a so-called “track-and-trace-label” ID-2-Label is attached, as a second identification marking, to the outside of the end face on the right of the dispatch carton for dispatching purposes, such as traceability. Upon arrival at the destination address, e.g., a dialysis center or a repeat-use dialysis patient, the dialysis modules D are removed individually from the dispatch carton and are sent for use in accordance with regulations.

Finally, it is noted that the description of the invention and the exemplified embodiments are fundamentally to be understood to be non-limiting with respect to a specific physical implementation. All features explained and illustrated in conjunction with individual embodiments of the invention can be provided in different combinations in the subject matter in accordance with the invention in order to achieve the advantageous effects thereof at the same time.

All the method steps can be implemented by devices which are suitable for carrying out the respective method step. All functions which are carried out by features can be a method step of a method. Furthermore, the components or means of the consumption device associated with the disposable, e.g., the medical apparatus, and the control system for performing a method can be distributed to a plurality of physical products or sub-assemblies.

The scope of protection of the present invention is set by the claims and is not limited by the features explained in the description or shown in the figures.

It is obvious to a person skilled in the art that the invention can be applied not only to protect dialysis methods, i.e., of medical usage applications in associated dialysis apparatuses against misuse and to safeguard against unauthorised use, but also to safeguard other technical system devices, in which appropriate disposables are used. Further examples of use of the proposed protection and monitoring against tampering or misuse by unauthorised third parties and auditing in system apparatuses for usage-relevant information (e.g., electronic health file) relate to the usage of membranes for reverse osmosis units. It is a basic concept of the invention, with regard to the disposables, for the manufacturer to provide a technical possibility, which is effective, specifically efficient and can also be used in a varied and flexible manner, for protecting the consumers or users and, where appropriate, the general public against the consequences of possible miscellaneous misuse and/or tampering and thus for the manufacturer to take further action to prevent such occurrences.

### LIST OF REFERENCE SIGNS

1 manufacturer
2 consumer
3 user
4 further participant
100 consumable
110 security element
150 concealing means
199 marking device
200 consumption device (e.g., medical apparatus)
250 code reading apparatus
300 database management control system
400 control system
D disposable (e.g., dialysis module)
ID-1-Code first identification code
ID-1-Label first identification marking
ID-2-Code second identification code
ID-2-Label second identification marking
Type product type/product usage property
ID-1-Code/
Type
first data relation
ID-1-Code
/ID-2-Code
second data relation
S101-S113 method steps

1-19. (canceled)

20. A disposable for usage in a medical apparatus, the disposable comprising a security element to protect against tampering or misuse by unauthorised third parties, wherein:
the security element has a first identification marking which materially forms a first identification code, and
the first identification marking is connected in a non-detachable manner to the disposable and is configured for uniquely identifying the disposable, and
wherein the first identification marking is partially or completely concealed with a concealing means which is detachable in a non-destructive-free manner.

21. The disposable of claim 20, further comprising:
a second identification marking which materially forms a second identification code,
wherein the second identification marking is unconcealed, clearly visible, and is used for at least temporary logistical use tracking, and
wherein the second identification marking is part of the concealing means.

22. A method for marking a disposable for usage in a medical apparatus to protect against tampering or misuse by unauthorised third parties, the method comprising:
generating, using information technology, a first identification code and storing, by a manufacturer, the consumable;
marking the disposable with a first identification marking which materially forms the first identification code;
allocating, using information technology, a product type and/or product usage property of the disposable to the first identification code to form a first data relation and storing by the manufacturer; and
concealing the first identification marking with a concealing means, wherein:
the first identification marking is partially or completely concealed; and
the concealing means is applied in such a manner that it can be detached in a non-destructive free manner from the first identification marking.

23. The method of claim 22, further comprising:
generating, using information technology, a second identification code and storing by the manufacturer;
marking the disposable with a second identification marking which materially forms the second identification code, wherein the second identification marking is applied in an unconcealed manner; and
allocating, using information technology, the second identification code to the first identification code to form a second data relation and storing by the manufacturer.

24. The method of claim 23, wherein the second identification marking for marking the disposable is part of the concealing means and is thus also used for partially or completely concealing the first identification marking.

25. The method of claim 23, wherein the concealing with the concealing means and/or the marking with the second identification marking is performed:
by means of a connection which can be detached in a destructive-free manner for the first identification marking; and/or
by means of reversible surface methods and/or multiple layer methods, by means of applying an adhesive cover layer, a cover layer which can be scratched off, an intermediate separation layer, a detachable adhesion layer, and/or by means of printing with reversible printing ink; and/or
in a quality control stage of the production of the disposable; and/or
in a packaging stage of the production of the disposable; and/or
in-line directly following on from and/or in an integral method stage with the step of marking with the first identification marking; and/or
on respective additional packaging of the packaging unit; and/or
in a clearly visible manner under transparent respective additional packaging of the packaging unit; and/or
wherein the second identification marking is in the form of a label which can be used for logistics purposes as a label configured for storing a transaction chain.

26. The method of claim 23, wherein the concealing means do not coincide with the second identification marking, and wherein the second identification marking is on a large pack including a plurality of disposables.

27. The method of claim 22, wherein the marking with the first identification marking is performed:
by means of a connection which can be detached in a non-destructive-free manner; and/or
by means of punching, lasing, printing, embossing, plating, coating, inlay methods, fixed adhesion, 3D printing, and/or sealing; and/or
in a quality control stage of the production of the disposable; and/or
in a packaging stage of the production of the disposable; and/or
on respective additional packaging of the packaging unit; and/or
in a clearly visible manner under transparent respective additional packaging of the packaging unit; and/or
by means of a connection which is non-detachable at least in an originally packaged and/or originally closed state of the disposable; and/or
in a region of the disposable which is concealed at least in the originally packaged and/or originally closed state and/or is not externally accessible.

28. The method of claim 22, further comprising:
removing the concealing means from the disposable at a usage location, wherein the first identification marking is exposed in a recognisable, clearly visible manner; and
authorising the usage and/or a respectively associated usage purpose of the disposable, wherein at least the first identification code is recognised.

29. The method of claim 28, wherein the second identification code is also recognised in advance of and/or at the same time as the recognition of the first identification code.

30. The method of claim 28, wherein at least the first identification code is read visually by a user or is recognised by means of a separate code reading apparatus and is transmitted to the consumption device and/or is automatically recognised by the consumption device.

31. The method of claim 28, wherein a connection of the concealing means to the disposable also serves to effect the removal of the concealing means during usage of the disposable.

32. The method of claim 28, further comprising disabling the disposable for usage after:
refusal of or failure to acquire authorisation relating to the non-compliance with a permissible usage purpose previously stored for a user, on the basis of a verification of a product type and/or product usage property of the disposable; and/or
an upper time limit allocated to the disposable on the basis of a shelf-life, and/or a time window after arriving at the usage location and/or after recognition of the first identification code; and/or
a compatibility of technical requirements relating to the correct usage of the disposable with the technical data of the consumption device,
wherein, in the event of a disabling action, information relating to an initiating reason and/or solution to a problem is also provided by the manufacturer.

33. The method of claim 28, wherein authorising is performed by comparing, using information technology, the first identification code recognised at the usage location with the first identification code which is generated and is stored by the manufacturer, the comparing comprising:
comparing with a copy of the generated first identification code previously stored in the consumption device; and/or
comparing with a copy of the generated first identification code previously stored with a user; and/or
comparing with a copy of the generated first identification code previously stored in a decentralised database of an authorised further participant; and/or
comparing and/or transmitting a previously stored copy of the generated first identification code by means of remote communication via an interface to a database of the manufacturer for managing at least the first identification code.

34. The method of claim 33, wherein authorising includes an information-technology use of a second data relation for taking into account information and/or a transaction chain collected as far the usage location.

35. The method of claim 33, wherein authorising includes an information-technology use of a first data relation for taking into account the product type and/or product usage property of the disposable.

36. The method of claim 35, wherein the information-technology use includes processing and/or storing in at least one block chain with linked transactions in a database management control system configured for guaranteeing documentation security between a plurality of distributed participants, the information-technology use comprising:
code-generating; storing, by the manufacturer, data from production and/or logistics; allocating to form the first data relation, allocating to form a second data relation; authorising; comparing; verifying; using the first data relation, using a second data relation; and disabling action; and/or
outputting information.

37. A control system which is configured to perform a method as claimed in claim 33, the control system comprising:
a control apparatus; and
a marking device configured to simultaneously and/or separately apply at least the first identification marking and/or the concealing means at a location of the manufacturer; and/or
a consumption device for usage of the consumable at the usage location of the user.

38. A computer program, loaded into a digital memory of a computer, which performs a method as claimed in claim 22.

39. A security element configured to be applied to a disposable for a usage application of a consumable and arranged for protecting the disposable against being tampered with or misused by unauthorised third parties, comprising:
a combination of a first identification marking, which materially forms a first identification code, and a second identification marking, which materially forms a second identification code, wherein
the first identification marking is arranged to form a non-detachable connection to the disposable and to provide a unique identification of the disposable in terms of a product type and/or product usage property of the disposable;
the first identification marking is partially or completely concealed with a concealing means which can be detached in a non-destructive-free manner;
the second identification marking is detachable and is configured for temporary use and logistical traceability; and
the second identification marking is identical to the concealing means.
